(19) United States
(12) Patent Application Publication
(10) Pub. No.: US 2023/0190194 A1
(43) Pub. Date: Jun. 22, 2023

(54) EAR-WEARABLE DEVICE OF MEASURING PHYSIOLOGICAL SIGNALS AND SYSTEM FOR MONITORING PHYSIOLOGICAL SIGNALS IN NON-FACE-TO-FACE

(71) Applicant: CMLAB CO., LTD., Seoul (KR)

(72) Inventors: Tae Ho Ha, Gyeonggi-do (KR); Joon Mo AN, Seoul (KR)

(21) Appl. No.: 17/556,117

(22) Filed: Dec. 20, 2021

(51) Int. Cl. A61B 5/00; A61B 5/11; A61B 5/01; A61B 5/1455

(52) U.S. Cl. CPC A61B 5/6815; A61B 5/0002; A61B 5/11; A61B 5/01; A61B 5/14551; A61B 2562/0271; A61B 2562/0219

(57) ABSTRACT

Ear-wearable device of measuring physiological signals disclosed. The ear-wearable device may include a body to be placed on a back of an ear, an ear hook, having one end coupled to the body and placed in front of the ear to fix the body to the ear, an oximeter, configured for measuring a saturation of percutaneous oxygen and having a detection surface disposed near the other end of the ear hook, an in-ear thermometer being connected to the body by a flexible cord, and a wireless transceiver, configured for wirelessly transmitting measurements of the oximeter and the in-ear thermometer.

### FIELD

The present invention relates to a non-face-to-face physiological signals measuring device that can be worn on a user's ear.

### BACKGROUND

In addition to telemedicine, epidemics such as COVID-19, which have recently become serious, are increasingly demanding the non-face-to-face provision of medical services. In order to provide medical services, medical professionals must accurately know the patient's condition. Physiological signals are the most basic and important information for diagnosing a patient's condition. Physiological signals such as body temperature, oxygen saturation, and pulse may be monitored in real time using a precise device provided in a healthcare facilities. However, when it is necessary to proceed with telemedicine or non-face-to-face treatment, it is not easy for the patient to have an aid of such a device.

A smart watch or sports ear set can measure the pulse based on oxygen saturation. However, since the physiological signals such as oxygen saturation is very sensitive to motion artifacts, even a slight movement during measurement may result in higher noise than a signal generated by the venous blood pressure wave. In terms of measuring the pulse precisely, these devices can provide the minimum information required for telemedicine or non-face-to-face health care, but cannot simultaneously measure other vital signs such as body temperature.

### SUMMARY

According to one aspect of the present invention, there is provided an ear-wearable device of measuring physiological signals. The ear-wearable device may include a body to be placed on a back of an ear, an ear hook, having one end coupled to the body and placed in front of the ear to fix the body to the ear, an oximeter, configured for measuring a saturation of percutaneous oxygen and having a detection surface disposed near the other end of the ear hook, an in-ear thermometer being connected to the body by a flexible cord, and a wireless transceiver, configured for wirelessly transmitting measurements of the oximeter and the in-ear thermometer.

In one embodiment, the ear hook may have S shape extending from the body and brings the oximeter into close contact with the back of the ear when worn.

In one embodiment, the ear hook may be rotatably coupled to the body.

In one embodiment, wherein the in-ear thermometer may include a thermometer housing coupled to the flexible code, a sensor assembly accommodated in the thermometer housing and having a temperature compensation function, and a protective cover coupled to the thermometer housing and defining an interior space in which the sensor assembly is accommodated.

In one embodiment, the sensor assembly may include an assembly housing, a temperature sensor accommodated in the assembly housing and configured for outputting a first measurement by receiving infrared radiation from a human body and a second measurement by measuring an internal temperature, and a thermoelectric module configured for increasing or decreasing the internal temperature.

In one embodiment, a pulse is calculated from measurements of the oximeter.

In one embodiment, the ear-wearable device may further include a gyro sensor configured for measuring acceleration to determine a user's movement.

According to another aspect of the present invention, there is provided a continuous non-face-to-face monitoring system. The continuous non-face-to-face monitoring system may include a plurality of ear-wearable devices configured for continuously measuring physiological signals and wirelessly transmitting measurements while worn by each of a plurality of users, and a monitoring device configured for collecting the measurements from the plurality of ear-wearable devices and processing physiological signals to detect an abnormal physiological signal. The ear-wearable devices may include a body to be placed on a back of an ear, an ear hook, having one end coupled to the body and placed in front of the ear to fix the body to the ear, an oximeter, configured for measuring a saturation of percutaneous oxygen and having a detection surface disposed near the other end of the ear hook, an in-ear thermometer being connected to the body by a flexible cord and a wireless transceiver, configured for wirelessly transmitting measurements of the oximeter and the in-ear thermometer.

In one embodiment, the ear hook may have S shape extending from the body and brings the oximeter into close contact with the back of the ear when worn.

In one embodiment, the ear hook may be rotatably coupled to the body.

In one embodiment, wherein the in-ear thermometer may include a thermometer housing coupled to the flexible code, a sensor assembly accommodated in the thermometer housing and having a temperature compensation function, and a protective cover coupled to the thermometer housing and defining an interior space in which the sensor assembly is accommodated.

In one embodiment, the sensor assembly may include an assembly housing, a temperature sensor accommodated in the assembly housing and configured for outputting a first measurement by receiving infrared radiation from a human body and a second measurement by measuring an internal temperature, and a thermoelectric module configured for increasing or decreasing the internal temperature.

In one embodiment, a pulse is calculated from measurements of the oximeter.

In one embodiment, the ear-wearable device may further include a gyro sensor configured for measuring acceleration to determine a user's movement.

### BRIEF DESCRIPTION OF ACCOMPANYING DRAWINGS

Hereinafter, embodiments of the invention will be described with reference to the accompanying drawings. For the purpose of easy understanding of the invention, the same elements will be referred to by the same reference signs. Configurations illustrated in the drawings are examples for describing the invention, and do not restrict the scope of the invention. Particularly, in the drawings, some elements are slightly exaggerated for the purpose of easy understanding of the invention. Since the drawings are used to easily understand the invention, it should be noted that widths, thicknesses, and the like of elements illustrated in the drawings might change at the time of actual implementation thereof.

FIG. 1 is a diagram exemplarily illustrating an ear-wearable device of measuring physiological signals;

FIG. 2 exemplarily illustrates steps of wearing the ear-wearable device of measuring physiological signals illustrated in FIG. 1;

FIG. 3 is an exploded perspective view exemplarily illustrating the ear-wearable device of measuring physiological signals illustrated in FIG. 1;

FIG. 4A, FIG. 4B and FIG. 4C exemplarily illustrates cross-sectional views of the sensor assembly illustrated in FIG. 3;

FIG. 5A and FIG. 5B exemplarily illustrate a coupling structure between a clip and a main body of the ear-wearable device of measuring physiological signals illustrated in FIG. 1;

FIG. 6 is a block diagram schematically illustrating the ear-wearable device of measuring physiological signals illustrated in FIG. 1; and

FIG. 7 exemplarily illustrates a continuous non-face-to-face monitoring system using the ear-wearable device of measuring physiological signals illustrated in FIG. 1.

### DETAILED DESCRIPTION

Embodiments which will be described below with reference to the accompanying drawings can be implemented singly or in combination with other embodiments. But this is not intended to limit the present invention to a certain embodiment, and it should be understood that all changes, modifications, equivalents or replacements within the spirits and scope of the present invention are included. Especially, any of functions, features, and/or embodiments can be implemented independently or jointly with other embodiments. Accordingly, it should be noted that the scope of the invention is not limited to the embodiments illustrated in the accompanying drawings.

If it is mentioned that an element is “connected to” or “coupled to” another element, it should be understood that still another element may be interposed therebetween, as well as that the element may be connected or coupled directly to another element. On the contrary, if it is mentioned that an element is “connected directly to” or “coupled directly to” another element, it should be understood that still another element is not interposed therebetween.

Terms such as first, second, etc., may be used to refer to various elements, but, these element should not be limited due to these terms. These terms will be used to distinguish one element from another element.

The terms used in the following description are intended to merely describe specific embodiments, but not intended to limit the invention. An expression of the singular number includes an expression of the plural number, so long as it is clearly read differently. The terms such as “include” and “have” are intended to indicate that features, numbers, steps, operations, elements, components, or combinations thereof used in the following description exist and it should thus be understood that the possibility of existence or addition of one or more other different features, numbers, steps, operations, elements, components, or combinations thereof is not excluded.

Elements of an embodiment described below with reference to the accompanying drawings are not limited to the corresponding embodiment, may be included in another embodiment without departing from the technical spirit of the invention. Although particular description is not made, plural embodiments may be embodied as one embodiment.

In describing the invention with reference to the accompanying drawings, like elements are referenced by like reference numerals or signs regardless of the drawing numbers and description thereof is not repeated. If it is determined that detailed description of known techniques involved in the invention makes the gist of the invention obscure, the detailed description thereof will not be made.

Terms such as ˜part, ˜unit, ˜module mean an element configured for performing a function or an operation. This can be implemented in hardware, software or combination thereof.

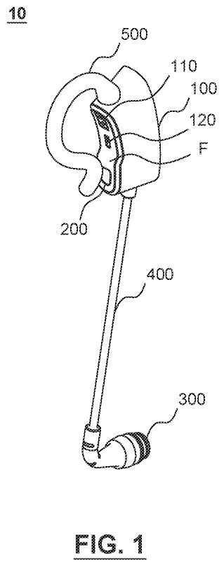

FIG. 1 is a diagram exemplarily illustrating an ear-wearable device of measuring physiological signals.

The ear-wearable device of measuring physiological signals 10 may measure at least one of physiological signals of a user. Here, the physiological signals that can be directly measured by the ear-wearable device 10 are a saturation of percutaneous oxygen, SpO₂, and a body temperature, and a pulse may be calculated using SpO₂ measurements. In addition to SpO₂ and body temperature, other physiological signals can also be measured in aid of an appropriate sensor.

The user may live while wearing the ear-wearable device of measuring physiological signals 10. Accordingly, the ear-wearable device 10 may continuously measure the physiological signals over a long period of time. Here, ‘continuous(ly)’ is a polysemantic word for mainly describing the state in which the ear-wearable device 10 is continuously worn, and even in the case that it is possible or sufficient for a certain physiological signal to be measured ‘periodically’ or ‘irregularly’, it may be described to be ‘continuously’ measured. For example, the user may wear the ear-wearable device 10 while sleeping as well as during daily work. Accordingly, it is possible to monitor changes in SpO₂, body temperature and/or pulse for hour, day or week.

An ear-worn type device, such as the ear-wearable device of measuring physiological signals 10, does not restrict the user's free use of his/her hands. Unlike SpO₂ that can be measured with a smart watch, body temperature must be measured while a thermometer is in contact with the ear or forehead or placed in a certain distance away. Therefore, when daily monitoring of body temperature is required, the user has to measure body temperature every predetermined time while carrying a thermometer. On the other hand, since an oximeter 200 for measuring SpO₂ in the ear-wearable device 10 is in close contact with the ear and an in-ear thermometer 300 remains inserted into the ear, the user's discomfort is considerably reduced. The ear-wearable device 10 may be provided not only to a user who can measure a physiological signal by itself, but also to a user who cannot measure a physiological signal by her/himself. In particular, the ear-wearable device 10 may be effective for continuously monitoring a patient with an epidemic that is likely to be infected upon contact.

Referring to FIG. 1, the ear-wearable device of measuring physiological signals 10 may include a body 100, the in-ear thermometer 300, a flexible cord 400, and an ear hook 500. The charging terminal 110, the switch 120, and the oximeter 200 exposed to the outside may be disposed on a front surface F of the body 100. The flexible cord 400 may electrically connect the body 100 and the in-ear thermometer 300. The ear hook 500 may couple the body 100, in particular, the oximeter 200 to the back of ear. Unlike other physiological signals, the oximeter 200 is vulnerable to motion artifacts and can only be measured when it is in close contact with the skin. The ear hook 500 made of an elastic material not only allows the oximeter 200 to be in close contact with the skin, but also to maintain the contact. In particular, since the ear is the body organ with the least movement, it is least affected by the movement of the hand or leg.

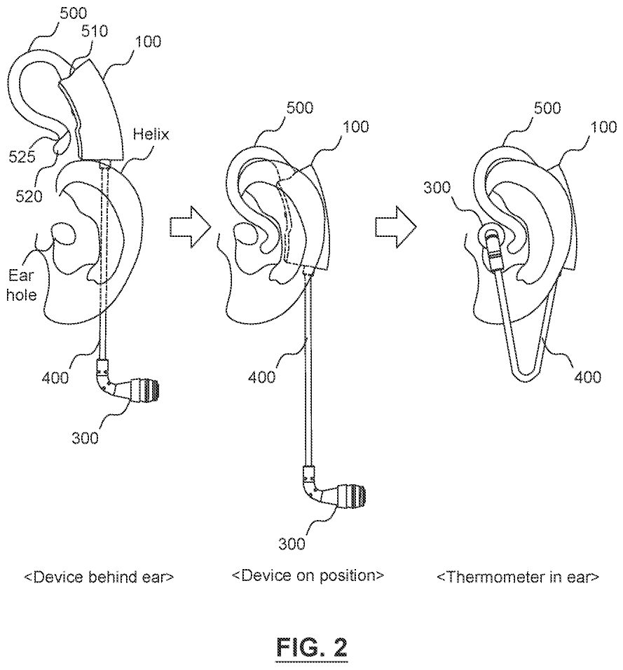

FIG. 2 exemplarily illustrates steps of wearing the ear-wearable device of measuring physiological signals illustrated in FIG. 1.

Ear hook 500 extends from one end 510 so that the other end 520 is spaced apart from the front surface F of the body 100. For example, the ear hook 500 may extend in a counterclockwise direction from the one end 510. The ear hook 500 may be formed of the elastic material to enable convenient wearing, and in particular, when wearing, the body 100 can be pulled toward the ear hook 500 to keep the close contact between the oximeter 200 and the skin. In one embodiment, for convenient wearing, the other end 520 of the ear hook 500 may be bent in a direction away from the front surface F to have S-shape. Due to this, the contact area 525 in contact with the ear may be located near the other end 520. Additionally, one end 510 of the ear hook 500 may be coupled to the upper surface or the front surface F of the main body 100.

The ear-wearable device of measuring physiological signals 10 may be worn by inserting an ear wheel into a gap between the contact area 525 of the ear hook 500 and the front surface F of the body 100. After being inserted, the ear hook 500 is located in front of the ear and the body 100 is located behind the ear. In FIG. 2, the oximeter 200 may partially protrude from the front surface F and be easily contacted with the back of the ear. Meanwhile, in order to increase the contact area with the back of the ear, at least a part or all of the front surface F may have a concave curved surface.

The position on the ear that comes into contact with the contact area 525 of the ear hook 500 should be spaced apart from the ear hole in the worn state. The portion extending from the other end 520 of the ear hook 500 to the contact area 525 should not interfere with the insertion of the in-ear thermometer 300 into the ear. When the contact area 525 or the other end 520 is close to the oxygen saturation detection sensor 200, the oxygen saturation detection sensor 200 may be more firmly in close contact with the ear. Accordingly, the position of the contact region 525 or the other end 520 may be adjusted according to the position of the oximeter 200.

After being fitted into the helix, by inserting the in-ear thermometer 300 suspended from the flexible cord 400 into the ear hole, the steps of wearing suitable for measuring physiological signals can be completed. The length of the flexible cord 400 may be determined not to pull the earlobe or the helix while being inserted into the ear.

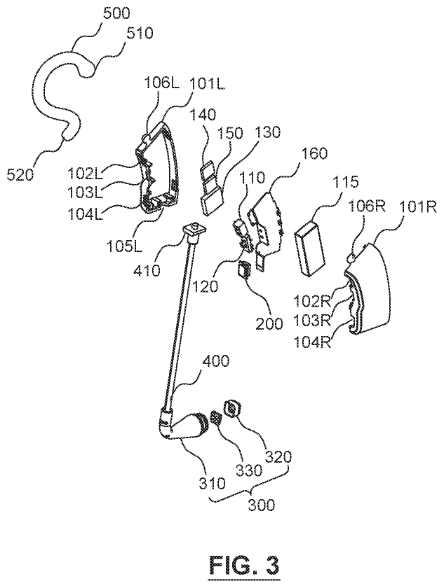

FIG. 3 is an exploded perspective view exemplarily illustrating the ear-wearable device of measuring physiological signals illustrated in FIG. 1.

The body 100 may include body housings 101L, 101R. In the front surface F of the body housings 101L, 101R, charging terminal holes 102L, 102R, switch holes 103L, 103R, and sensor holes 103L, 103R may be formed at positions corresponding to the charging terminal 110, the switch 120, and the oximeter 200. An internal space is defined by the body housings 101L and 101R, and at least some of the components implementing the function of the ear-wearable device of measuring physiological signals 10 may be accommodated in the defined internal space.

The charging terminal 110 is electrically connected to the rechargeable battery 115 and receives power for charging the battery 115 from the outside. If a wireless charger is adopted, the charging terminal 110 and the charging terminal holes 102L and 102R may be omitted.

The switch 120 controls the operation of the ear-wearable device of measuring physiological signals 10. Power may be supplied to each component from the battery 115 by the switch 120. Since the switch 120 is disposed on the front surface F, it becomes difficult to turn off the ear-wearable device 10 while being worn. Accordingly, the ear-wearable device 10 may not be turned off while being worn by intentional manipulation or a mistake.

The oximeter 200 has a detection surface on which a plurality of LEDs and photo diodes are disposed. The LEDs may include red and near infrared LEDs. The detection surface of the oximeter 200 may be exposed through the sensor holes 103L and 103R. The oximeter 200 measures the light absorption of arterial blood using red light and near-infrared light. SpO₂ can be calculated using functional hemoglobin HbO₂ that transports oxygen bound in the lungs to tissues and dysfunctional hemoglobin Hb that does not transport oxygen. HbO₂ and Hb exhibit different optical properties, namely, different absorbances, in the optical band between about 600 nm and about 1,000 nm. At about 660 nm red light, HbO₂ has a relatively greater absorbance than Hb. Using this, the absorbances can be measured by alternatingly irradiating red light and near-infrared light at regular intervals. From these measurements, the oxygen saturation can be calculated, and the pulse can be calculated from the oxygen saturation.

The body housings 101L and 101R may accommodate the battery 115, the microprocessor 130, the wireless transceiver 140, and the gyro sensor 150. Power lines from the battery 115 to each component and signal lines between the sensors 200, 330 and the microprocessor 130 may be provided by the printed circuit board 160. The charging terminal 110 and the switch 120 may be fixed to the printed circuit board 160.

The wireless transceiver 140 transmits physiological signals or receives control commands in one or more communication protocols under the control of the microprocessor 130. Here, the wireless transceiver 140 may communicate with, for example, a base station in a mobile communication network such as W-CDMA, a Wifi router in LAN, or any communication device supporting short-range wireless communication such as NFC, Bluetooth, Zigbee, Wifi-Direct and so on (hereinafter base station, Wifi router, and communication device will be collectively referred to as an access point). When only short-range wireless communication is possible, the wireless transceiver 140 may be connected to the access point through a mobile communication terminal such as a smart phone, for example. The wireless transceiver 140 converts the electrical signal output from the microprocessor 130 or the sensors 200, 300 into a wireless signal and transmits it to an external device through the access point, and receives the wireless signals from the access point.

The gyro sensor 150 measures an acceleration generated in the ear-wearable device of measuring physiological signals 10. A movement of the user wearing the ear-wearable device 10, for example, walking or running may be detected through changes in the acceleration. The duration time of user's movement may be recorded and managed as exercise time. Meanwhile, since the motion artifacts increases while the user is moving, the oximetry may be temporarily paused. When the user collapses or falls, the gyro sensor 150 may measure an acceleration instantaneously generated in the direction of gravity.

The in-ear thermometer 300 may include a thermometer housing 310, a protective cover 320, and a sensor assembly 330. The sensor assembly 330 is accommodated in an internal space defined by the thermometer housing 310 and the protective cover 320. The protective cover 320 may at least partially include an opening or optically transparent medium to allow passage of infrared light emitted from the inner ear.

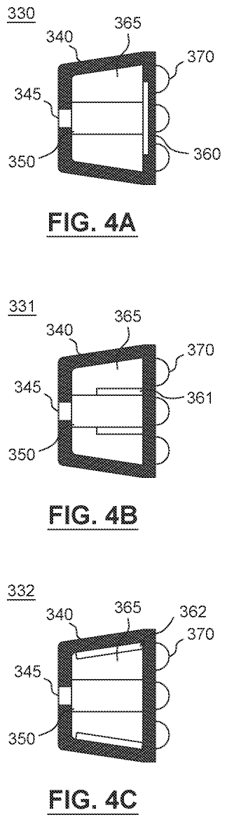

Meanwhile, the sensor assembly 330 may have a temperature compensation function. The temperature compensation function maintains the temperature of the sensor itself within the normal operating range, thereby reducing the occurrence of measurement errors due to changes in ambient temperature. Various embodiments of the sensor assembly 330 are described in detail with reference to FIG. 4.

One end of the flexible cord 400 is coupled to the in-ear thermometer 300, and the vicinity of the other end is coupled to a cord strain relief bushing 410. The cord strain relief bushing 410 is inserted into grooves for bushing 105L, 105R formed in the lower portions of the body housings 101L, 101R. The flexible cord 400 may include an electrical conductor therein, for example, a wire. The flexible cord 400 provides power from the battery 115 to the in-ear thermometer 300 and conveys control commands and measurements between the in-ear thermometer 300 and the microprocessor 130.

FIG. 4A, FIG. 4B and FIG. 4C exemplarily illustrates cross-sectional views of the sensor assembly illustrated in FIG. 3.

The sensor assemblies 330, 331, 332 have a temperature compensation function. The sensor assemblies 330, 331, 332 may include an assembly housing 340, a temperature sensor 350, and thermoelectric modules such as TEC (Thermoelectric cooler) 360, 361, 362. The temperature sensor includes an infrared sensor configured for receiving infrared radiation emitted from the inner ear, a sensor housing that fixes the infrared sensor so that a light-receiving surface of the infrared sensor is disposed substantially perpendicular to an optical axis, and an internal sensor configured for measuring a temperature of the infrared sensor itself or a temperature around the infrared sensor (hereinafter, collectively referred to as ‘internal temperature’). The assembly housing 340 may include a window 345 positioned on the optical axis of the temperature sensor 350. The sensor housing may be a cylindrical housing with one end open or protected by an optically transparent medium. The sensor housing is electrically insulated from the infrared sensor, and may be formed of a material having high thermal conductivity.

The temperature sensor 350 measures infrared radiations emitted from the human body to output a first measurement signal and the internal temperature to output a second measurement signal. As an example of the temperature sensor, the thermopile sensor STP9CF55 manufactured by Sunshine Technologies of China includes a thermopile sensor that outputs the first measurement signal and an internal sensor that outputs the second measurement signal, for example, a thermistor. The infrared sensor is a negative temperature coefficient (NTC) sensor whose resistance value decreases as the temperature indicated by the received infrared radiation increases, and the internal sensor may be a positive temperature coefficient (PTC) sensor whose resistance value increases as the internal temperature increases.

The temperature sensor 350 is thermally coupled to the thermoelectric modules 360, 361, 362. The thermoelectric modules 360, 361, 362 may be driven by electricity to remove heat from the temperature sensor 350 (namely, cooling) or transfer heat to the temperature sensor 350 (namely, heating). Additionally, the sensor assemblies 330, 331, 332 may further include a metal member (not shown) for increasing heat exchange efficiency between the thermoelectric modules 360, 361, 362 and the temperature sensor 350. Hereinafter, the conventional method of correcting the measured body temperature using the internal temperature and the temperature compensation process according to the present invention will be described.

A thermopile sensor, which is an example of an infrared sensor, may output a different first measurement signal according to an operating temperature, even when infrared rays representing the same temperature are received. Conventionally, the internal temperature obtained through the second measurement signal is used to correct the first measurement signal itself or the body temperature calculated from the first measurement signal. However, in order to correct the first measurement signal and/or the body temperature calculated therefrom, a conversion formula or conversion table is required for each thermopile sensor. The conversion formula or conversion table inevitably includes an error within a certain range, and due to the influence of the PVT (Process, Voltage, Temperature), even the same type of thermopile sensor may output the first measurement signal having a relatively large error when it is out of an appropriate temperature range. In addition, the conversion formula or conversion table should be different for each thermopile sensor.

When the sensor assemblies 330, 331, 332 are turned on, the internal sensor of the temperature sensor 350 outputs the second measurement signal representing the current internal temperature T_{current}. The microprocessor 130 calculates a difference T_{target}−T_{current} by comparing the preset appropriate internal temperature T_{target} and the current internal temperature T_{current}. If the difference is positive, an increase in the internal temperature, namely, heating is required, and if the difference is negative, a decrease in the internal temperature, namely, cooling is required. The appropriate internal temperature T_{target} may be room temperature, such as 25 degrees Celsius. As described above, the thermopile sensor is manufactured in consideration of operation at room temperature, and may output a relatively precise first measurement signal than at other operating temperatures.

The microprocessor 130 may control the thermoelectric modules 360, 361, 362 to generate or remove heat according to the difference T_{target}−T_{current}. The microprocessor 130 controls thermoelectric modules 360, 361, 362 to heat or cool one of both surfaces of the facing the temperature sensor 350 by adjusting the direction, magnitude, and application time of the direct current applied to the thermoelectric modules 360, 361, 362 according to the temperature control command. When the difference T_{target}−T_{current} is large, a relatively large current is applied for a certain period of time to induce a relatively rapid temperature rise. The heat generated by the thermoelectric modules 360, 361, 362 is transferred to the temperature sensor 350 to increase the internal temperature. The internal sensor measures the increased internal temperature and outputs the second measurement signal, and the microprocessor 130 compares the current internal temperature T_{current} calculated using the second measurement signal with an appropriate internal temperature T_{target}. When the difference T_{target}−T_{current} decreases, the magnitude of the current applied to the thermoelectric modules 360, 361, 362 is reduced, so that the rate of temperature increase may be controlled. When cooling is required, that is, when the difference T_{target}−T_{current} is negative, the microprocessor 130 cools the surface facing the temperature sensor 350 among both surfaces of the thermoelectric module 360, 361, 362 by changing the direction of the current applied to the thermoelectric module 360, 361, 362. The surface facing the medium temperature sensor 350 is cooled. This lowers the internal temperature.

Referring to FIG. 4A, the sensor assembly 330 may include a plate-shaped thermoelectric module 360 disposed on the inner bottom of the assembly housing 340. The thermoelectric module 360 may be in contact with or may not come into contact with the temperature sensor 350. When disposed in contact with the other end of the temperature sensor 350, the thermoelectric module 360 may directly heat or cool the sensor housing of the temperature sensor 350. When disposed to be spaced apart from the other end of the temperature sensor 350, the thermoelectric module 360 heats or cools the inner space 365 of the assembly housing 340, thereby indirectly heating or cooling the infrared sensor and/or the sensor housing. Through this, the internal temperature may be increased or decreased.

In FIGS. 4B and 4C, the sensor assemblies 331, 332 may include the thermoelectric modules 361, 362 formed to have a curved surface disposed on a lateral surface of the temperature sensor 350. In FIG. 4B, the thermoelectric module 361 may contact the lower outer surface of the temperature sensor 350 to directly heat or cool the sensor housing of the temperature sensor 350. In FIG. 4C, the thermoelectric module 362 may be disposed on the lower inner surface of the assembly housing 340 to heat or cool the inner space 365, thereby indirectly heating the infrared sensor and/or the sensor housing. Through this, the temperature of the infrared sensor itself or the ambient temperature may be increased or decreased.

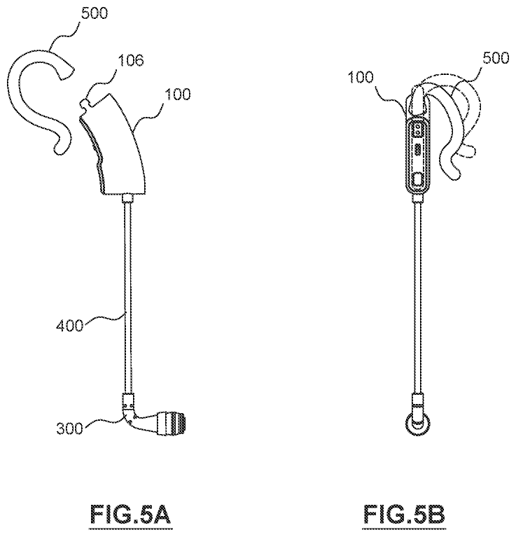

FIG. 5A and FIG. 5B exemplarily illustrate a coupling structure between a clip and a main body of the ear-wearable device of measuring physiological signals illustrated in FIG. 1.

Referring to FIG. 5A and FIG. 5B together, a receiving socket 511 may be formed at one end of the ear hook 500, the receiving socket 511 may accommodate a coupling ball 106 therein. The coupling ball 106 may be disposed on the upper surface of the body 100. The receiving socket 511 and the coupling balls 106L and 106R constitute a ball joint, whereby the ear hook 500 can rotate around the direction in which the coupling ball 106 extends from the upper surface. Unlike the way of wearing illustrated in FIG. 2, the user may wear the ear-wearable device of measuring physiological signals 10 by rotating the ear hook 500 in a clockwise or counterclockwise direction, placing the body 100 on the back of the ear, and then rotating the ear hook 500 in the opposite direction.

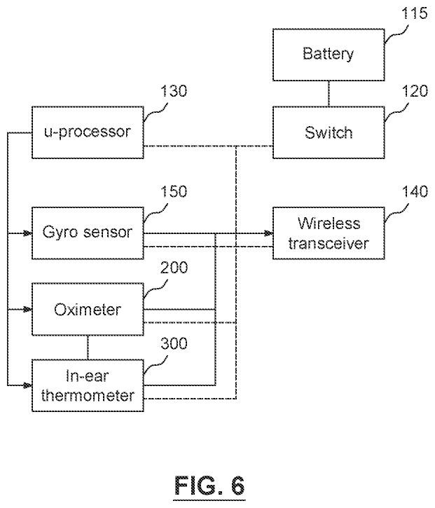

FIG. 6 is a block diagram schematically illustrating the ear-wearable device of measuring physiological signals illustrated in FIG. 1.

Referring to FIG. 6, the ear-wearable device of measuring physiological signals 10 may include the microprocessor 130, the oximeter 200, the in-ear thermometer 300, the gyro sensor 150, and the wireless transceiver 140. The operation of the ear-wearable device 10 may be initiated by the switch 120.

The microprocessor 130 may control the operation of the oximeter 200, the in-ear thermometer 300, and the wireless transceiver 140. For example, the microprocessor 130 may drive the sensors 200, 300 at regular time intervals to measure the physiological signals. The time intervals of the oximeter 200 and the in-ear thermometer 300 may be the same or different. The time interval may be an interval set by default or an interval set by a measurement control command received from the outside. The microprocessor 130 may determine whether the measurement of the physiological signal is within a predetermined normal range. In addition, the microprocessor 130 may allow the wireless transceiver 140 to transmit the measurements to the outside.

The microprocessor 130 may measure the user's movement through the gyro sensor 150. For example, the microprocessor 130 may determine that the user is in a walking or running from a repetitive acceleration change pattern measured by the gyro sensor 150. The user's movement may be temporarily stored and then transmitted to the outside. As another example, the microprocessor 130 may temporarily pause measuring SpO₂ while the user moves. As still another example, when the instantaneous acceleration in the direction of gravity is measured, the microprocessor 130 determines that the user is collapsing or falling, and may transmit an emergency signal to the outside through the mobile communication terminal communicating with the wireless transceiver 140.

Additionally or alternatively, the microprocessor 130 may control the sensor assembly 330 of the in-ear thermometer 300 to perform the temperature compensation operation. The microprocessor 130 receives the second measurement signal indicating the internal temperature from the sensor assembly 330 and drives the thermoelectric modules 360, 361, 362 so that the second measurement signal does not deviate from the predetermined normal temperature range.

The oximeter 200 may measure SpO₂ and calculate a pulse by using the measured SpO₂. Meanwhile, the oximeter 200 measures only SpO₂, and the pulse rate may be calculated by the microprocessor 130. The oximeter 200 may use optical properties of hemoglobin showing different absorbance when red light and near-infrared light are irradiated. The SpO₂ measurement may include not only an AC component but also a DC component. Unlike the AC component that represents the actual SpO₂, DC noise is generated by other living tissues other than hemoglobin, and various methods to remove it, such as Beer-Lambert, Peak and Valley, MASIMO, etc., can be applied.

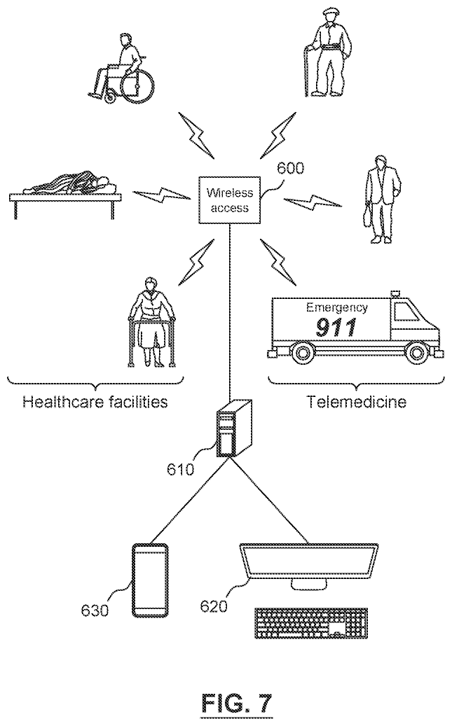

FIG. 7 exemplarily illustrates a continuous non-face-to-face monitoring system using the ear-wearable device of measuring physiological signals illustrated in FIG. 1.

The fire detection system using video surveillance can easily manage large buildings or large areas. This is because images from multiple places can be centrally monitored in one place. However, medical equipment used in hospitals is mainly equipped with wired communication functions, limiting movement after wearing, and thus failing to provide a way to monitor patients out of their seats. In particular, it is not easy to measure the condition of a patient outside the scope of the medical professionals.

The ear-wearable device of measuring physiological signals 10 enables continuous and non-face-to-face monitoring of a plurality of patients accommodated in a healthcare facility. Hospitals are equipped with medical professional and medical equipment capable of caring for multiple patients. However, it is practically impossible to monitor the condition by simultaneously measuring the physiological signals of all patients. In particular, continuous monitoring is essential as the condition of epidemic patients can deteriorate rapidly. The ear-wearable device 10 may be worn for every patient accommodated in the healthcare facility, and an abnormal physiological signal among the measured physiological signals may be automatically detected and notified to the medical professional.

The ear-wearable device of measuring physiological signals 10 enables provision of telemedicine services to people who are not accommodated in healthcare facilities. Telemedicine services can be provided not only to people with diseases that require constant care, but also to emergency patients who are being transported to healthcare facilities. Even in daily life, physiological signals may be continuously measured and used as basic information for medical evaluation.

Referring to FIG. 7, the continuous non-face-to-face monitoring system may include the ear-wearable device of measuring physiological signals 10, a monitoring device for collecting and processing physiological signals 610, and terminals 620, 630. The terminal may be an electronic information processing device having a communication function, such as a personal computer, a laptop computer, a smart phone, a pad and so on used by medical professional. The ear-wearable device 10 transmits the measurements of physiological signals to the monitoring device 610 through the access point 600. Meanwhile, the monitoring device 610 may also transmit a measurement control command to the ear-wearable device 10 through the access point 600. The monitoring device 610 is an electronic information processing device such as a computer/server connected to the terminals 620 and 630 through a wired or wireless communication network. The monitoring device 610 includes a central processing unit such as CPU or GPU, a memory such as RAM, ROM and/or flash memory, a communication interface such as LAN adapter, and a storage medium such as a flash drive. Operations such as collection, analysis, and provision of physiological signals may be implemented by the central processing unit executing a program loaded on the memory or stored in the storage medium.

The monitoring device for collecting and processing physiological signals 610 collects one or more physiological signals from the plurality of ear-wearable device of measuring physiological signals 10. The collected physiological signals are processed separately for each ear-wearable device 10 or a person who wears the same. Here, the processing may include not only a process of analyzing the physiological signal to determine whether it is abnormal, but also a process of storing the physiological signal before or after processing. The range for determining normal/abnormal may be set differently for each physiological signal.

The above description of the invention is exemplary, and those skilled in the art can understand that the invention can be modified in other forms without changing the technical concept or the essential feature of the invention. Therefore, it should be understood that the above-mentioned embodiments are exemplary in all respects, but are not definitive.

The scope of the invention is defined by the appended claims, not by the above detailed description, and it should be construed that all changes or modifications derived from the meanings and scope of the claims and equivalent concepts thereof are included in the scope of the invention.

What is claimed is:

1. An ear-wearable device of measuring physiological signals, comprising:
a body to be placed on a back of an ear;
an ear hook, having one end coupled to the body and placed in front of the ear to fix the body to the ear;
an oximeter, configured for measuring a saturation of percutaneous oxygen and having a detection surface disposed near the other end of the ear hook;
an in-ear thermometer being connected to the body by a flexible cord; and
a wireless transceiver, configured for wirelessly transmitting measurements of the oximeter and the in-ear thermometer.

2. The ear-wearable device of claim 1, wherein the ear hook has S shape extending from the body and brings the oximeter into close contact with the back of the ear when worn.

3. The ear-wearable device of claim 1, wherein the ear hook is rotatably coupled to the body.

4. The ear-wearable device of claim 1, wherein the in-ear thermometer comprises:
a thermometer housing coupled to the flexible code;
a sensor assembly accommodated in the thermometer housing and having a temperature compensation function; and
a protective cover coupled to the thermometer housing and defining an interior space in which the sensor assembly is accommodated.

5. The ear-wearable device of claim 4, wherein the sensor assembly comprises:
an assembly housing;
a temperature sensor accommodated in the assembly housing and configured for outputting a first measurement by receiving infrared radiation from a human body and a second measurement by measuring an internal temperature; and
a thermoelectric module configured for increasing or decreasing the internal temperature.

6. The ear-wearable device of claim 1, wherein a pulse is calculated from measurements of the oximeter.

7. The ear-wearable device of claim 1 further comprising a gyro sensor configured for measuring acceleration to determine a user's movement.

8. A continuous non-face-to-face monitoring system, comprising:
a plurality of ear-wearable devices configured for continuously measuring physiological signals and wirelessly transmitting measurements while worn by each of a plurality of users; and
a monitoring device configured for collecting the measurements from the plurality of ear-wearable devices and processing physiological signals to detect an abnormal physiological signal,
wherein the ear-wearable devices comprises:
a body to be placed on a back of an ear;
an ear hook, having one end coupled to the body and placed in front of the ear to fix the body to the ear;
an oximeter, configured for measuring a saturation of percutaneous oxygen and having a detection surface disposed near the other end of the ear hook;
an in-ear thermometer being connected to the body by a flexible cord; and
a wireless transceiver, configured for wirelessly transmitting measurements of the oximeter and the in-ear thermometer.

9. The continuous non-face-to-face monitoring system of claim 8, wherein the ear hook has S shape extending from the body and brings the oximeter into close contact with the back of the ear when worn.

10. The continuous non-face-to-face monitoring system of claim 8, wherein

11. The continuous non-face-to-face monitoring system of claim 8, wherein the in-ear thermometer comprises:
a thermometer housing coupled to the flexible code;
a sensor assembly accommodated in the thermometer housing and having a temperature compensation function; and
a protective cover coupled to the thermometer housing and defining an interior space in which the sensor assembly is accommodated.

12. The continuous non-face-to-face monitoring system of claim 11, wherein the sensor assembly comprises:
an assembly housing;
a temperature sensor accommodated in the assembly housing and configured for outputting a first measurement by receiving infrared radiation from a human body and a second measurement by measuring an internal temperature; and
a thermoelectric module configured for increasing or decreasing the internal temperature.

13. The continuous non-face-to-face monitoring system of claim 8, wherein a pulse is calculated from measurements of the oximeter.

14. The continuous non-face-to-face monitoring system of claim 8 further comprising a gyro sensor configured for measuring acceleration to determine a user's movement.
